(19) United States
(12) Patent Application Publication
(10) Pub. No.: US 2005/0107586 A1
(43) Pub. Date: May 19, 2005

(54) Lp354 Mammalian secreted protein

(76) Inventors: Eric Wen Su, Carmel, IN (US); Yu Zhi, Indianapolis, IN (US)

Correspondence Address: ELI LILLY AND COMPANY;PATENT DIVISION, P.O. BOX 6288, INDIANAPOLIS, IN, 46206-6288, US

(21) Appl. No.: 10/481,029

(22) PCT Filed: Apr. 17, 2002
(86) PCT No.: PCT/US02/12182

Related U.S. Application Data
(60) Provisional application No. 60/308,882, filed on Jul. 31, 2001.

(51) Int. Cl. C07H 21/04; C07K 14/705; C07K 14/47

(52) U.S. Cl. 530/350; 435/69.1; 435/320.1; 435/325; 536/23.5

(57) ABSTRACT

The present invention overcomes problems in the prior art of updating/upgrading remote control to include functions and/or abilities nor originally planned when the remote control was being designed/manufactured. A set-top device itself can be an updated device which can be bundled with an outdated remote control. The set-top can provide a remote teacher display or such a display can be made available from a distance via the internet or satellite. In such a case, a remote control can be used to select a specific on-screen display button from the remote teacher. The corresponding remote control code is then taught to a learning remote control by a blaster which serves as a substitute for a teaching remote control in order to teach and/or update/upgrade the learning remote control.

### FIELD OF THE INVENTION

The present invention relates to the teaching of learning remote controls, and more particularly to the teaching of learning remote controls from a blaster in order to update/upgrade the enabled functions, information, or codes of the remote control.

### BACKGROUND TO THE INVENTION

With the proliferation of remote controllable devices in a home, e.g., video cassette recorders (VCRs), video disc players (DVD), and several television sets, each of which usually requires different programmed formats and codes, a different dedicated remote control is required to control each device. In order to reduce such a proliferation of remote controls, learning remote controls have been introduced. A single learning remote control typically can store control sequences for controlling various devices. Learning remote controls are shown in U.S. Pat. Nos, 4,866,434, 4,802,114 and 4,623,887 wherein a reconfigurable remote control device has the ability to learn, store and repeat remote control codes taught by teaching remote controls.

In order to program a learning remote control, a teacher remote control is positioned In a communicating spatial relationship with the learning remote control and the learning remote control is placed in a learning mode, i.e., certain switches are operated in order to establish a memory location dedicated to storing data relating to particular functions. As a result, infrared data signals are transmitted from the dedicated remote control to the learning remote control whereby the data sequence is received and stored. The stored data can be accessed by a switch dedicated to a particular function, and the stored corresponding control sequence is then transmitted via infrared energy to the device to be controlled, in place of the original bundled remote control. This functional ability applies for the control keys of the learning remote control. The data sequences for several different controlled devices can be stored associated with keys dedicated to the various devices.

In a brief summary of a prior art learning sequence, control codes can be stored locally In a memory of the learning remote. After entering a command to the teaching remote control, the corresponding control sequences for that device are transmitted to the learning remote control. The IR signal is received by the learning remote control, translated into data, and stored in its memory. The stored data can also include address data which causes the control sequence data to be stored at remote control memory locations that are accessible by particular keys. That is, predesignated keys on the teaming remote control can access data at predetermined address locations in the memory where particular control sequence data designated by the address date has been stored, thus allowing the user to control various different devices by means of particular common switches. For example, different brands of VCRs can be controlled using the same standard control switches on the remote control for on/off, channel switching, time set, etc. Alternatively the control sequence data transmitted to the learning remote can load the learning remote control memory at a predetermined memory location, and the remote control switches can access the required control sequences due to the memory address locations where the required control sequences are stored.

Other techniques for storing a program for invoking specialized functions have been tried, such as the use of dedicated number sequences published in popular media such as newspapers, to allow a user to key in those number sequences into a special remote control, e.g. VCR PLUS®. The special IR remote control is then placed to be in communication with the IR remote control receiver of the VCR so that at the appropriate time, the remote control will activate and cause the VCR to record the desired program. This does not require that users know how to program their VCR or learn how to set the VCR clock. However this Is limited and is specialized to the programming of a VCR to start/stop recording of a designated channel at particular times.

Recent developments have provided other requirements for controlling a device, often a VCR, from external devices, e.g., cable television set-top boxes, satellite TV set-top boxes, or high definition (HDTV) set-top boxes for viewing HD digital programming on an analog TV receiver. In such cases, an electronic program guide (EPG) can be provided on a display, e.g., a TV receiver, and the user makes a selection with an IR remote control. It is often required that these external devices be able to control the VCR to stop/start recording of a designated channel at particular times, much like the VCR PLUS® set-up discussed above. To accomplish this, the set-top boxes can be provided with an appropriate output port and the ability to use software and/or hardware to drive an “IR blaster” (an Infrared emitter).

The IR blaster is connected by a cable from the set-top box and Is located to be able to communicate with the IR remote controllable receiver in order to control the receiver, e.g., VCR, a cable television decoder box. For such control, the IR blaster substitutes for the controlled device's bundled remote control, and thus the set-top box has the ability to drive the blaster LED(s) and is provided with the software/hardware for providing the IR blaster signal with the correct parameters so that the controllable device, e.g., VCR, television receiver, will accept the signal as if the signal is coming from its own bundled remote control. An example of a system using an IR blaster is shown in U.S. Pat. Nos. 6,151,059, 6,057,874, and IR blasters are provided with REPLAY® and TIVO® personal television servers to control VCRs, cable set-top boxes, etc. After the remote control selection of the command from a displayed list on the television screen, the associated control sequence data can be transmitted at the appropriate time by the IR blaster to, e.g. a VCR, TV, cable television set-top box or decoder, etc.

It is a problem to provide the user with advanced codes for a remote control if the codes did not exist when the remote control, bundled with a device, was designed or manufactured. The bundled remote control might be a learning remote in anticipation of a manufacturer later providing new codes, or the manufacturer might provide a non-learning remote and the owner must purchase an accessory learning remote to capture the codes provided by the on-screen display. For such a learning remote, some fixes for such a problem are: 1) posting the new code on a website, or 2) doing a “rolling change” so that newly manufactured remotes would have a special feature, e.g., a simultaneous keypress of two buttons on the remote would send out a different code for directly controlling the set-top box. The weakness of the first approach is that the user is forced to go to a website to find out what the code is and then find instructions/files about how to program their learning remote to use the latest codes. This can be daunting for someone who is not technically inclined. Additionally, the instruction book for the remote control would have to be updated and be code might be “hidden” (not have a dedicated key) and this would require special directions to the user. The weakness of the second approach is that if the new remotes have a hidden code and the old remotes do not, old remote users might think that something was wrong with their remote if they should happen to find out about new codes available with the new remotes.

### SUMMARY OF THE INVENTION

The present invention overcomes problems in the prior art of updating/upgrading remote controls to include functions and/or abilities not originally planned when the remote control was being designed/manufactured. A set-top device itself can be an updated device which can be bundled with an outdated remote control. The set-top box can provide a remote teacher display or such a display can be made available from a distance via the internet or satellite. In such a case, a remote control can be used to select a specific on-screen display button from the remote teacher, The corresponding remote control code is then taught to a learning remote control by a blaster driven by the device In such a case, the device is able to drive a blaster which serves as a substitute for a teaching remote control in is order to teach and/or update/upgrade the learning remote control.

### BRIEF DESCRIPTION OF THE DRAWINGS

Referring now to the drawings:

FIG. 1 shows an exemplary remote teaching menu according to aspects of the present invention.

FIG. 2 shows an exemplary arrangement according to aspects of the present invention.

### DETAILED DESCRIPTION OF THE PREFERRED EMBODIMENT

The present device is an HDTV set-top box for receiving high definition television pictures, for viewing on an analog television receiver, The HDTV pictures can be provided via satellite using a satellite set-top receiver, provided by a cable television provider which may or may not be available via cable television set-top box which may also serve as a decoder, or be provided by broadcast and received with a local antenna. These items are all remote controllable and the present invention applies to any one of them with the present HDTV set-top device being exemplary. Additionally, the present discussion can include any device with an IR blaster output such as a Gemstar T , a VCR, or a Personal Video Recorder like ReplayTV®, TIVO® or UltimateTV®.

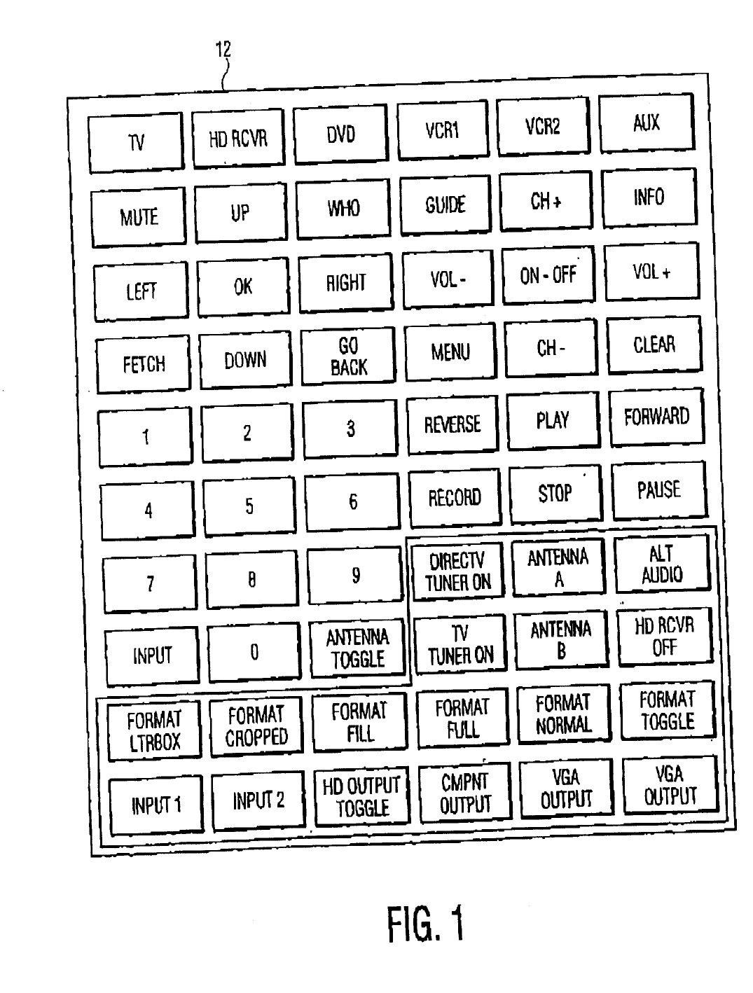

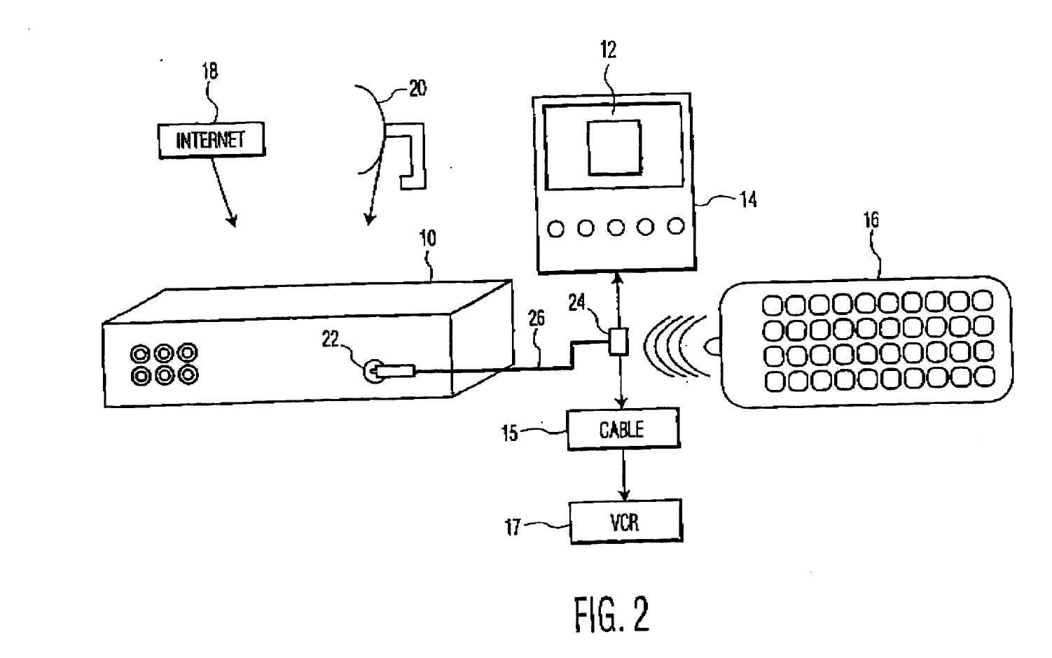

Referring to FIG. 2, a set-top device 10 menu is presented in an on-screen display 12 shown on a television receiver 14. An exemplary on-screen display 12 is shown in FIG. 1 which is a teaching display for navigation and selection by a remote control 16, which is supplied with device 10. Device 10 may also receive such an on-screen display 12 from Internet 18 or satellite antenna 20 for presentation on television receiver 14. In other words, a remote on screen display (OSD) for teaching a learning remote, is received from a distant source. The data representing an OSD is received from the internet via a modem or other connection, or satellite antenna and receiver, or other source, and displayed in response to activation of a teaching mode.

Device 10 is provided with an IR blaster output jack 22 for connection of an IR blaster 24 via a cable 26. Blaster 24 can be positioned so that its output IR signal can be received by a VIC 17 or cable television set-top box 15 as if controlled by the remote control for the respective VCR 17 or cable television set-top box 15. This blaster control can be provided with a choice of commands or functions chosen from an appropriate on-screen display 12 for the particular device.

In the exemplary embodiment, IR remote control 16 is a learning remote which may be bundled (provided with) device 10 or it can be an after acquired accessory. The on-screen remote teaching display 12 can be programmed into device 10, or it can be provided by receipt of an internet page from 18, or via a satellite receiver via antenna 20. The providing of an on screen display (OSD) such as an electronic program guide is known in the prior art as used In the TIVO® and REPLAY®devices discussed above, or the DTC 100 HDTV set-top box manufactured by Thomson Consumer Electronics, Inc.® of Indianapolis Indiana, USA.

The problems of updatng/upgrading of remote controls are discussed above. The present solution to the prior art updating/upgrading problem of a remote control is to provide an on-screen display in the menu system for the user to send specific teaching codes via the IR blaster to an awaiting learning remote control 16, which learns the code, i.e.. device 10 and IR blaster 24 are enabled to blast programming and/or codes to learning remote control 16 which is then able to control a VCR 17, e.g., to toggle VIC power, change VIC channel and/or start recording, or to have a cable set-top box 15 change channel. Thus, device 10 and IR blaster 24 are additionally programmed and enabled to serve as a teaching remote control to blast/teach learning remote 16 either with completely new information, codes and/or functions or with updated/upgraded information, codes and/or functions.

As non-limiting examples, referring to the next to the last row of FIG. 1, remote control 16 can be taught to provide various complex functions, i.e., selection of picture formats, or to execute relatively simple commands, i.e., to toggle between formats, even if the remote control was not originally programmed to do so. In this way, using such a remote teaching on-screen display, learning remote 16 can be provided with updated/upgraded information, codes, and/or functions which were not available at the time of design or manufacture of remote control 16.

Additionally, the present invention can also provide IR codes for functions that are available in the product but just could't be fit on the remote control which has a limited number of buttons. This is especially true for home enthusiasts who desire discrete IR codes for functions to put their equipment into a known state. Instead of a single toggle button on the remote to toggle (switch) between, for example, related functions, e.g., input source selections, home enthusiasts may desire a separate discrete IR code assigned to each separate function. In the example of input selection, the home enthusiast might create a button on his/her learning remote that puts the TV into a known state by sending a discrete code to tune directly to INPUT2 of the TV, i.e., the enthusiast would prefer not to press a toggle key multiple times to and have to toggle thru INPUT 3, INPUT 4, and INPUT 1 just to get to INPUT 2.

Such discrete codes are also useful for creating strings of commands called macros in order to place equipment into a known state.

Although the present embodiment is disclosed in connection with an IR blaster, it Is within the contemplation of the present invention that RF blasters are included and that the claims are applicable to such RF blasters. As used herein, RF is defined as electromagnetic energy at longer wavelengths than the far Infrared.

1. A reconfigurable remote control system comprising:
a learning remote control for operating a remote controllable device, and
a teaching device for teaching and reconfiguring the learning remote control,
the teaching device being a blaster.

2. A system of programming a remote control comprising:
a learning remote control,
transmitting means via an transmitter to the remote control data for control of a selected device;
the transmitting means being a blaster.

3. A remote control system comprising a learning remote control and a teaching means for teaching the learning remote control, the teaching means comprising a blaster.

4. A set-top box television system comprising:
a set-top box which is remote controllable and is provided with a blaster for controlling an external device, and a remote control for remote controlling the set-top box,
the set-top box's own remote control being teachable by transmission from the blaster.

5. An electronic device having a remote control for remotely controlling the electronic device, the electronic device being able to remotely teach its own remote control.

6. The electronic device of claim 5 wherein the electronic device is able to teach its own remote control via a blaster.

7. A system for updating the functionality of a system having a remote control, comprising:
an electronic device which Is remote controllable;
a remote control for remotely controlling the electronic device, and
a blaster which is drivable by the electronic device,
the remote control being updatable/upgradable by programming provided by the electronic device with codes which are conveyed to the remote control by the blaster.

8. The system of claims 1, 2, 3, 4, 6 and 7 wherein the blaster is an IR blaster.


SEQUENCE LISTING (14 sequences)

SEQ ID NO: 1 - protein, 90 aa, Homo sapiens
MEAASLSVATAGVALALGPETSSRTRDPKPERDTRFDPERRRPAGPRAALLCLHGPGGDAASAADRRHFPVEPAGSGHHPAGPCLPPRAA

SEQ ID NO: 2 - DNA, 273 bp, Homo sapiens
atggaggccgctagcctttcagtggccaccgccggcgttgcccttgccctgggacccgagaccagcagcaggacccgggaccccaagcccgagagggatactcggttcgaccccgagcggcgccgtcctgccgggccgagggccgcccttctctgtcttcacggtcctggtggtgacgctgctagtgctgctgatcgccgccactttcctgtggaacctgctggttccggtcaccatcccgcgggtccgtgccttccaccgcgtgccgcataa

SEQ ID NO: 3 - protein, 74 aa, Homo sapiens
LGPETSSRTRDPKPERDTRFDPERRRPAGPRAALLCLHGPGGDAASAADRRHFPVEPAGSGHHPAGPCLPPRAA

SEQ ID NO: 4 - DNA, 225 bp, Homo sapiens
ctgggacccgagaccagcagcaggacccgggaccccaagcccgagagggatactcggttcgaccccgagcggcgccgtcctgccgggccgagggccgcccttctctgtcttcacggtcctggtggtgacgctgctagtgctgctgatcgccgccactttcctgtggaacctgctggttccggtcaccatcccgcgggtccgtgccttccaccgcgtgccgcataa

SEQ ID NO: 5 - DNA, 354 bp, Homo sapiens
gccccctgccctggagctgcgatcccaagcgccatggaggccgctagcctttcagtggccaccgccggcgttgcccttgccctgggacccgagaccagcagcaggacccgggaccccaagcccgagagggatactcggttcgaccccgagcggcgccgtcctgccgggccgagggccgcccttctctgtcttcacggtcctggtggtgacgctgctagtgctgctgatcgccgccactttcctgtggaacctgctggttccggtcaccatcccgcgggtccgtgccttccaccgcgtgccgcataacttggtggcctcgacggccgtctcggacgaactagtggcagcgctggc

SEQ ID NO: 6 - protein, 370 aa, Homo sapiens
MEAASLSVATAGVALALGPETSSGTPSPRGILGSTPSGAVLPGRGPPFSVFTVLVVTLLVLLIAATFLWNLLVPVTIPRVRAFHRVPHNLVASTAVSDELVAALAMPPSLASELSTGRRRLLGRSLCHVWISFDALCCPAGLGNVAAIALGRDGAITRHLQHTLRTRSRASLLMIALARVPSALIALAPLLFGRGEVCDARLQRCQVSREPSYAAFSTRGAFHLPLGVVPFVYRKIYEAAKFRFGRRRRAVLPLPATMQVKEAPDEAEVVFTAHCKATVSFQVSGDSWREQKERRAAMMVGILIGVFVLCWIPFFLTELISPLCACSLPPIWKSIFLWLGYSNSFFNPLIYTAFNKNYNNAFKSLFTKQR

SEQ ID NO: 7 - DNA, 731 bp, Homo sapiens
gggatccggagcccaaatcttctgacaaaactcacacatgcccaccgtgcccagcacctgaattcgagggtgcaccgtcagtcttcctttccccccaaaacccaaggacaccctcatgatctcccggactcctgaggtcacatgcgtggtggtggacgtaagccacgaagaccctgaggtcaagttcaactggtacgtggacgcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaacccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccgcgactctagaggat

SEQ ID NO: 8 - DNA, 85 bp, Homo sapiens
gcgcctcgagatttccccgaaatctagatttccccgaaatgatttccccgaatgatttccccgaaatatctgccatctcaattag

SEQ ID NO: 9 - DNA, 27 bp, Homo sapiens
gcggcaagctttttgcaaagcctaggc

SEQ ID NO: 10 - DNA, 270 bp, Homo sapiens
ctcgagatttccccgaaatctagatttccccgaaatgattccccgaaatgatttccccgaaatatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 11 - DNA, 32 bp, Artificial Sequence
  Synthetic Construct
gcgctcgagggatgacagcgatagaaccccgg

SEQ ID NO: 12 - DNA, 31 bp, Artificial Sequence
  Synthetic Construct
gcgaagcttcgcgactccccggatccgcctc

SEQ ID NO: 13 - DNA, 27 bp, Artificial Sequence
  Synthetic Construct
gcggcaagctttttgcaaagcctaggc

SEQ ID NO: 14 - DNA, 256 bp, Homo sapiens
ctcgaggggactttcccggggactttccggggactttccgggactttccatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt